(19) United States
(12) Patent Application Publication
(10) Pub. No.: US 2010/0062052 A1
(43) Pub. Date: Mar. 11, 2010

(54) PEPTIDE-BASED COMPOUNDS AS NEW INHIBITORS OF METALLOECTOPEPTIDASES, COMPOSITIONS COMPRISING SAID COMPOUNDS AND THEIR PHARMACEUTICAL AND COSMETIC USES

(75) Inventor: Philippe Bernard, La Ferte Saint Aubin (FR)

Correspondence Address: CANTOR COLBURN, LLP, 20 Church Street, 22nd Floor, Hartford, CT, 06103, US

(73) Assignee: GREENPHARMA, Saint Beauzire (FR)

(21) Appl. No.: 12/524,955

(22) PCT Filed: Feb. 4, 2008
(86) PCT No.: PCT/IB08/01328

§ 371 Date: Jul. 29, 2009

Related U.S. Application Data
(60) Provisional application No. 60/899,033, filed on Feb. 2, 2007.

(51) Int. Cl. A61K 9/127; C07K 5/10; C07K 7/06; C07K 5/08; C07K 5/06; A61K 38/05; A61K 38/06; A61K 38/07; A61K 38/08; A61K 9/16; A61K 8/18; A61Q 17/04; A61Q 1/10; A61Q 9/00; A61Q 11/00

(52) U.S. Cl. 424/450; 530/331; 530/330; 514/19; 514/18; 514/17; 424/490; 424/59; 424/70.7; 424/73; 424/49

(57) ABSTRACT

The invention relates to a compound that inhibits a metallo-ectopeptidase, selected in the group consisting of x-aa2-aa1-z, x-aa3-aa2-aa1-z, x-aa4-aa3-aa2-aa1-z and x-aa5-aa4-aa3-aa2-aa1-z, wherein: aa1 and aa4, independently from each other, represent an arginine amino acid, an histidine amino acid, an asparagine amino acid or a lysine amino acid, aa2 represents a serine amino acid, a glycine amino acid, a threonine amino acid or an alanine amino acid, aa3 represents a phenylalanine amino acid, a leucine amino acid, an isoleucine amino acid, a tyrosine amino acid or a tryptophan amino acid, aa5 represents a glutamine amino acid, a glutamic acid amino acid, an asparagines amino-acid or a lysine amino acid, x and z, independently from each other, represent: —H; —OH1 SH; —a halogen atom, —an amino group, —an alkyl, haloalkyl or heteroalkyl group containing from 1 to 30 carbon atoms, linear or branched, —an alkenyl or alkynyl group containing from 2 to 30 carbon atoms, linear or branched, —a cycloalkyl, cycloakenyl, cycloalkynyl or an heterocyclic group containing from 3 to 30 carbon atoms, linear or branched, —one or more aryl or heteroaryl groups containing from 3 to 10 carbon atoms per cycle, —a alkoxy, thioalkyl, sulfonylalkyl, aminoalkyl containing from 1 to 30 carbon atoms, linear or branched, —one or more heterocyclic group, containing from 5 to 10 carbon atoms per cycle, said groups being optionally substituted by one or more halogen atoms, alkyl groups, hydroxy groups, alkoxy groups, aryloxy groups, acyloxy groups, carbamoyloxy groups, carboxy groups, mercapto groups, alkylthio groups, acylthio groups, arylthio groups, aryl groups, heterocyclic groups, heteroaryl groups or amino groups.

The present invention relates to new peptide-based compounds as new inhibitors of metallo-ectopeptidases and compositions comprising said compounds. The present invention also relates to their pharmaceutical and cosmetic uses.

Mammalian zinc ectopeptidases, located at the surface of cells in nervous and systemic tissues, play important roles in turning off neural and hormonal peptide signals at the cell surface, notably those processing sensory information. Among the neuronal peptide-signals are enkephalins, which are potently and rapidly inactivated in vivo by two ecto-enzymes, NEP (Neutral EndoPeptidase; EC 3.4.24.11) and AP-N (AminoPeptidase-N; EC 3.4.11.2). Enkephalins are involved in regulating pain and nociception in the body. Enkephalins play a crucial role in the dynamic control of neurotransmission pathways of pain and in the modulation of cerebral structure activity governing, among others, motivation and the adaptive equilibrium of emotional states. Their action is specifically transmitted, like that of morphine (an alkaloid opiate), via μ- and ∂-opioid membrane receptors. The identification of the mechanisms which control the upstream regulation of enkephalin signals is of fundamental significance to physiological and therapeutic studies because of the importance of the biological constants regulated by the endogenous opioid system.

An inhibitor of enkephalin activating zinc ectopeptidases in humans, named opiorphin, has been recently discovered (Wisner et al., 2006). It is a QRFSR peptide that inhibits the human neutral ecto-endopeptidase, hNEP, and the human ecto-aminopeptidase, hAP-N. Opiorphin displays potent analgesic activity in chemical and mechanical pain models by activating endogenous opioid-dependent transmission. The function of opiorphin is closely related to the bovine spinorphin peptide (Nishimura et al., 1993; Yamamoto et al., 2002) and to the rat sialorphin peptide (Rougeot et al., 2003).

European application EP 1 577 320 describes the QRFSR peptide and its use in the prevention and treatment of pain. This application also describes a YQRFSR peptide and a Glp-RFSR peptide, in which Glp is pyroglutamate.

Inventors have now identified new peptide-based compounds, derived from the QRFSR peptide. These new compounds exhibit inhibitory properties against metallo-ectopeptidases, in particular hNEP and/or hAP-N.

Advantageously, the peptide-based compounds of the invention enhance the bioavailability of the compounds, because of their structure. They may also advantageously have other biological properties, such as anti-inflammatory, anti-oxidant or antiseptic properties.

### DESCRIPTION OF THE INVENTION

The present invention relates to a compound that inhibits a metallo-ectopeptidase, preferably the Neutral EndoPeptidase (EC 3.4.24.11) and/or the AminoPeptidase-N (EC 3.4.11.2), said compound being selected in the group consisting of x-aa2-aa1-z, x-aa3-aa2-aa1-z, x-aa4-aa3-aa2-aa1-z and x-aa5-aa4-aa3-aa2-aa1-z, wherein:

aa1 and aa4, independently from each other, represent an arginine amino acid, an histidine amino acid, an asparagine amino acid or a lysine amino acid,

aa2 represents a serine amino acid, a glycine amino acid, a threonine amino acid or an alanine amino acid,

aa3 represents a phenylalanine amino acid, a leucine amino acid, an isoleucine amino acid, a tyrosine amino acid or a tryptophan amino acid,

aa5 represents a glutamine amino acid, a glutamic acid amino acid, an asparagines amino-acid or a lysine amino acid,

and wherein x and z, independently from each other, represent:
H;
OH, SH;
a halogen atom,
an amino group,
an alkyl, haloalkyl or heteroalkyl group containing from 1 to 30 carbon atoms, linear or branched,
an alkenyl or alkynyl group containing from 2 to 30 carbon atoms, linear or branched,
an acyl group containing from 1 to 30 carbon atoms, saturated or unsaturated, linear or branched,
a cycloalkyl, cycloakenyl, cycloalkynyl or an heterocyclic group containing from 3 to 30 carbon atoms, linear or branched,
one or more aryl or heteroaryl groups containing from 3 to 10 carbon atoms per cycle,
a alkoxy, thioalkyl, sulfonylalkyl, aminoalkyl containing from 1 to 30 carbon atoms, linear or branched,
one or more heterocyclic group, containing from 5 to 10 carbon atoms per cycle,
   said groups being optionally substituted by one or more halogen atoms, alkyl groups, hydroxy groups, alkoxy groups, aryloxy groups, acyloxy groups, carbamoyloxy groups, carboxy groups, mercapto groups, alkylthio groups, acylthio groups, arylthio groups, aryl groups, heterocyclic groups, heteroaryl groups or amino groups.

The peptide is preferably selected from the group consisting of x-SR-z, x-FSR-z, x-RFSR-z and x-QRFSR-z.

More preferably, z is H or NH₂ and x is a saturated or unsaturated, linear or branched fatty acid containing from 2 to 24 carbon atoms and possibly substituted by one or more heteroatoms.

The invention also relates to a composition comprising the above-mentioned compound, in association with an acceptable carrier. Said composition may be a cosmetic or pharmaceutical topical composition.

Another object of the present invention is the use of the above-defined compound for the preparation of a medicament for treating of preventing pain, or in a topical cosmetic formulation.

Finally, the invention relates to a process of cosmetically treating human skin or a mucous membrane comprising applying an effective amount of the topical composition to said skin or mucous membrane.

### Peptide-Based Compounds

The compounds of the invention are selected in the group consisting of

x-aa2-aa1-z (I)

x-aa3-aa2-aa1-z (II)

x-aa4-aa3-aa2-aa1-z (III)

x-aa5-aa4-aa3-aa2-aa1-z (IV)

wherein:

aa1 and aa4, independently from each other, represent an arginine amino acid, an histidine amino acid or a lysine amino acid,

aa2 represents a serine amino acid, a glycine amino acid, a threonine amino acid or an alanine amino acid,

aa3 represents a phenylalanine amino acid, a leucine amino acid, an isoleucine amino acid, a tyrosine amino acid or a tryptophan amino acid,

aa5 represents a glutamine amino acid, a glutamic acid amino acid, an asparagines amino-acid or a lysine amino acid.

The compounds of the invention have at least a peptide moiety. “Peptide” refers to a polymer in which the monomers are alpha amino acids joined together through amide bonds. According to the invention, the peptide moiety is two to five amino acid monomers long. Preferably, the peptide moiety is three to five amino acid monomers long.

Amino acid residues in peptides are abbreviated as follows:

| | | |
|---|---|---|
| Ala | or A | is alanine |
| Arg | or R | is arginine |
| Asn | or N | is asparagine |
| Asp | or D | is aspartic acid |
| Cys | or C | is cysteine |
| Glu | or E | is glutamic acid |
| Gln | or Q | is glutamine |
| Gly | or G | is glycine |
| His | or H | is histidine |
| Ile | or I | is isoleucine |
| Leu | or L | is leucine |
| Lys | or K | is lysine |
| Met | or M | is methionine |
| Phe | or F | is phenylalanine |
| Pro | or P | is proline |
| Ser | or S | is serine |
| Thr | or T | is threonine |
| Trp | or W | is tryptophan |
| Tyr | or Y | is tyrosine |
| Val | or V | is valine |

According to the present invention, the amino acids of the peptide moiety may either be in a D- or a L-configuration. A D-configuration is preferred if the compound of the invention is to not to be degraded enzymatically. The D-amino acid can be at either the N-terminus or the C-terminus. Advantageously, the D-amino acid is at the N-terminus.

In the above-mentioned formulas, x and z, independently from each other, represent:

H;

OH, SH;

a halogen atom,

an amino group,

an alkyl, haloalkyl or heteroalkyl group containing from 1 to 30 carbon atoms, linear or branched,

an alkenyl or alkynyl group containing from 2 to 30 carbon atoms, linear or branched,

an acyl group containing from 1 to 30 carbon atoms, saturated or unsaturated, linear or branched,

a cycloalkyl, cycloakenyl, cycloalkynyl or an heterocyclic group containing from 3 to 30 carbon atoms, linear or branched,

one or more aryl or heteroaryl groups containing from 3 to 10 carbon atoms per cycle,

a alkoxy, thioalkyl, sulfonylalkyl, aminoalkyl containing from 1 to 30 carbon atoms, linear or branched,

one or more heterocyclic group, containing from 5 to 10 carbon atoms per cycle,

said groups being optionally substituted by one or more halogen atoms, alkyl groups, hydroxy groups, alkoxy groups, aryloxy groups, acyloxy groups, carbamoyloxy groups, carboxy groups, mercapto groups, alkylthio groups, acylthio groups, arylthio groups, aryl groups, heterocyclic groups, heteroaryl groups or amino groups.

“Halogen atom” means F, Cl, Br, or I. In one embodiment, the halogen atom is F, Cl, or Br, more particularly Cl or F, and most particularly F.

“Alkyl group” means a chain of 1 to 40 carbon atoms, particularly 1 to 30 carbon atoms. Lower alkyl groups can include alkyl groups such as methyl and ethyl. Monovalent hydrocarbon groups may have a straight-chain or branched-chain structure. In one embodiment, the branched alkyl groups have one or two branches, particularly one branch. Alkyl groups are saturated. One to four hydrogen atoms bonded to carbon atoms in the alkyl group may have been replaced with one or more substituents. As an example, x represents a palmitoyl, a stearyl or a palmitoyl residue.

According to the present invention, “substituent” may include, but are not limited to, halogen atoms, halogenated hydrocarbon groups, alkyl groups (e.g., methyl, ethyl, propyl, and butyl), hydroxy groups, alkoxy groups (e.g., methoxy, ethoxy, propoxy, butoxy, and pentoxy), aryloxy groups (e.g., phenoxy, chlorophenoxy, tolyloxy, methoxyphenoxy, benzyloxy, alkyloxycarbonylphenoxy, and acyloxyphenoxy), acyloxy groups (e.g., propionyloxy, benzoyloxy, and acetoxy), carbamoyloxy groups, carboxy groups, mercapto groups, alkylthio groups, acylthio groups, arylthio groups (e.g., phenylthio, chlorophenylthio, alkylphenylthio, alkoxyphenylthio, benzylthio, and alkyloxycarbonylphenylthio), aryl groups (e.g., phenyl, tolyl, alkoxyphenyl, alkoxycarbonylphenyl, and halophenyl), heterocyclic groups, heteroaryl groups, and amino groups (e.g., amino, mono- and di-alkanyl-amino groups of 1 to 3 carbon atoms, methylphenylamino, methylbenzylamino, alkanylamido groups of 1 to 3 carbon atoms, carbamamido, ureido, and guanidino).

“Alkenyl group” and “alkynyl group” mean a chain of 2 to 30 carbon atoms, having one or more double bonds, one or more triple bonds, or combinations thereof. Alkenyl and alkynyl groups are unsaturated. One or more hydrogen atoms bonded to carbon atoms in the alkenyl or alkynyl group may have been replaced with one or more substituents. As an example, x represents a oleoyl, a linolenyl, an arachidyl or a myristoyl residue

“Acyl group” means a chain of 1 to 30 carbon atoms, saturated or unsaturated, which may have a straight-chain or branched-chain structure. Acyl groups usually derived from carboxylic acids. As an example, x represents a palmitoyl, a stearyl or a palmitoyl residue.

“Cycloalkyl group”, “cycloakenyl group” and “cycloalkynyl group” mean a saturated or unsaturated carbocyclic (or hydrocarbon) ring. Carbocyclic groups are monocyclic, or are fused, spiro, or bridged bicyclic ring systems. Monocyclic carbocyclic groups contain 3 to 10 carbon atoms, particularly 4 to 7 carbon atoms, and more particularly 5 to 6 carbon atoms in the ring. Bicyclic carbocyclic groups contain 8 to 12 carbon atoms, and more particularly 9 to 10 carbon atoms in the ring. Examples of carbocyclic groups include, but are not limited to, cyclopentyl, cyclohexyl, cyclohexenyl, cycloheptyl, and cyclooctyl. The carbocyclic groups are particularly cyclopentyl, cyclohexyl, and cyclooctyl. According to the present invention, carbocyclic groups may be substituted or unsubstituted.

“Heterocyclic group” means a saturated or unsaturated ring structure containing carbon and 1 to 4 heteroatoms in the ring. The attachment point for heterocyclic groups may be at one or more carbon atoms, one or more nitrogen atoms (if present) or a combination of carbon and nitrogen atoms. Heterocyclic groups are monocyclic, or are fused or bridged bicyclic ring systems. Monocyclic heterocyclic groups contain 3 to 10 member atoms (i.e., including both carbon atoms and at least 1 heteroatom), particularly 4 to 7 member atoms, and more particularly 5 to 6 member atoms in the ring. Bicyclic heterocyclic groups contain 8 to 12 member atoms, particularly, 9 or 10 member atoms in the ring. Examples of heterocyclic groups include 1,3-dioxolane, 1,3-dioxane, piperizyl, morpholinyl, tetrahydrofuranyl, tetrahydropyranyl, and piperidyl. According to the present invention, heterocyclic groups may be substituted or unsubstituted.

“Aryl group” means an aromatic group having a monocyclic ring structure or fused bicyclic ring structure. Monocyclic aromatic groups contain 3 to 10 carbon atoms, particularly 5 to 7 carbon atoms, and more particularly 5 to 6 carbon atoms in the ring. Bicyclic aromatic groups contain 8 to 12 carbon atoms, particularly 9 or 10 carbon atoms in the ring. Bicyclic aromatic groups include groups wherein only one ring is aromatic or where both rings are aromatic. Aromatic groups may be substituted or unsubstituted. One suitable aromatic group is phenyl.

“Heteroaryl group” means an aromatic ring containing carbon and 1 to 4 heteroatoms in the ring. Heteroaromatic groups are monocyclic or fused bicyclic rings. Monocyclic heteroaromatic groups contain 3 to 10 member atoms (i.e., carbon and heteroatoms), particularly 5 to 7 member atoms, and more particularly 5 to 6 member atoms in the ring. Bicyclic heteroaromatic rings contain 8 to 12 member atoms and more particularly 9 or 10 member atoms in the ring. Examples of heteroaromatic groups include, but are not limited to, thienyl, thiazolyl, purinyl, pyrimidyl, pyridyl, and furanyl. In one embodiment, heteroaromatic groups include thienyl, furanyl, and pyridyl. One particularly suitable heteroaromatic group is thienyl. According to the present invention, heteroaryl groups may be substituted or unsubstituted.

In one embodiment, according to the present invention, x and z may, similarly of independently, be a lipid component, a carbohydrate component, a terpene, a terpenoid, a carotenoid, a vitamin component, a rosmarinic acid residue or a flavonoid.

According to the present invention, the lipid component can be any lipid-containing component, such as a lipopeptide, fatty acid, phospholipid, steroid, or a lipidated amino acids and glycolipids such as Lipid A derivatives. In one embodiment, the lipid component is a fatty-acid, that is a monocarboxylic acid with an unbranched aliphatic chain, which is either saturated or unsaturated. Examples of saturated fatty acid include, but are not limited to, butanoic acid, hexanoic acid, octanoic acid decanoic acid, dodecanoic acid, tetradecanoic acid, hexadecanoic acid (or palmitic acid), octadecanoic acid (or stearic acid), eicosanoic acid, docosanoic acid and tetracosanoic acid. Omega-3 fatty acids, omega-6 fatty acids and omega-9 fatty acids are examples of polyunsaturated fatty acids. Examples of unsaturated fatty acids include, but are not limited to, myristoleic acid, palmitoleic acid, oleic acid, linoleic acid, alpha-linolenic acid, arachidonic acid, eicosapentaenoic acid, erucic acid and docosahexaenoic acid. Preferred lipids for use as the lipid component of the peptide-based compound of the invention include palmitic acid (or palmitate) and stearic acid (or stearate).

The carbohydrate component of the peptide-based compound can be any component that contains a carbohydrate. Examples of suitable carbohydrate components include oligosaccharides, polysaccharides and monosaccharides, and glycosylated biomolecules (glycoconjugates) such as glycoproteins, glycopeptides, glycolipids, glycosylated amino acids, DNA, or RNA. Glycosylated peptides (glycopeptides) and glycosylated amino acids contain one or more carbohydrate moieties as well as a peptide or amino acid.

The carbohydrate component of the peptide-based compound of the invention includes a carbohydrate that contains one or more saccharide monomers. For example, the carbohydrate can include a monosaccharide, a disaccharide or a trisaccharide; it can include an oligosaccharide or a polysaccharide. An oligosaccharide is an oligomeric saccharide that contains two or more saccharides and is characterized by a well-defined structure. A well-defined structure is characterized by the particular identity, order, linkage positions (including branch points), and linkage stereochemistry ([alpha], [beta]) of the monomers, and as a result has a defined molecular weight and composition. An oligosaccharide typically contains about 2 to about 20 or more saccharide monomers. A polysaccharide, on the other hand, is a polymeric saccharide that does not have a well defined structure; the identity, order, linkage positions (including brand points) and/or linkage stereochemistry can vary from molecule to molecule. Polysaccharides typically contain a larger number of monomeric components than oligosaccharides and thus have higher molecular weights. The term “glycan” as used herein is inclusive of both oligosaccharides and polysaccharides, and includes both branched and unbranched polymers. When the carbohydrate component contains a carbohydrate that has three or more saccharide monomers, the carbohydrate can be a linear chain or it can be a branched chain. In a preferred embodiment, the carbohydrate component contains less than about 15 saccharide monomers; more preferably in contains less than about 10 saccharide monomers.

According to an embodiment of the present invention, x and z may also be, independently from each other, a terpene. Terpenes are derived biosynthetically from units of isoprene, which has the molecular formula C₅H₈. The basic molecular formulas of terpenes are multiples of that, (C₅H₈)n where n is the number of linked isoprene units. Terpenes are classified according to the number of isoprene units: monoterpenes (n=2), sesquiterpenes (n=3), diterpenes (n=4), triterpenes (n=6) and tetraterpenes (n=8). Examples of monoterpenes include, but are not limited to, pinene, nerol, citral, camphor, geraniol and limonene. Examples of sesquiterpenes include, but are not limited to, nerolidol and farnesol. Squalene is an example of a triterpene.

According to the present invention, terpenes may be hydrocarbons, but also oxygen-containing compounds such as alcohols, aldehydes or ketones (terpenoids). The term “terpenoid”, for purposes of the present invention, is intended to cover terpenes and oxygen containing derivatives thereof having at least one C₅H₈ unit which may have one or more points of unsaturation and/or be part of a cyclic unit within the chemical structure.

The carotenoids belong to the category of tetraterpenoids (i.e. they contain 40 carbon atoms). Structurally they are in the form of a polyene chain which is sometimes terminated by rings. Carotenoids with molecules containing oxygen, such as lutein and zeaxanthin, are known as xanthophylls. The unoxygenated (oxygen free) carotenoids such as alpha-carotene, beta-carotene and lycopene are known as carotenes. Carotenes typically contain only carbon and hydrogen.

The vitamin component of the peptide-based compound can be either water-soluble or fat-soluble. Water-soluble vitamins include B vitamins, notably vitamin B1 (thiamin) and vitamin B6 (pyridoxine, pyridoxal, and pyridoxamine) and vitamin C. Fat-soluble vitamins include vitamins A, D (notably D3 vitamin, D2 vitamin or derivatives thereof), E (tocopherol and tocotrienol) and K.

According to an embodiment of the present invention, x and z may also be a rosmarinic acid residue. Rosmarinic acid is known as having anti-inflammatory properties.

According to an embodiment of the present invention, x and z may also be, independently from each other, a flavonoid. According to the present invention, the flavonoids may derived from a 2-phenylchromen-4-one (2-phenyl-1,4-benzopyrone) structure, from a 3-phenylchromen-4-one (3-phenyl-1,4-benzopyrone) structure (commonly known as isoflavonoid) or from a 4-phenylcoumarine (4-phenyl-1,2-benzopyrone) structure (commonly known as a neoflavonoid). Flavonoids are most commonly known for their antioxidant activity. Examples of flavonoid according to the present invention include, but are not limited to, quercetin and genistein.

According to the present invention, when the compound is x-QRFSR-z and z is H, then x is different from H. Additionally, when the compound is x-QRFSR-z and z is H, then x is different from a tyrosine amino acid. Finally, when the compound is x-RFSR-z and z is H, then x is different from a pyroglutamate residue. Accordingly, the peptide-based compound of the invention is different from the QRFSR peptide, as well as the YQRFSR and Glp-RFSR peptides/compounds.

One or more linkers are optionally used for assembly of the compound of the invention. In on embodiment, the linker is a bifunctional linker that has functional groups in two different places, preferably at a first and second end, in order to covalently link the peptide moiety of the compound with another component of the peptide. A bifunctional linker can be either homofunctional (i.e., containing two identical functional groups) or heterofunctional (i.e., containing two different functional groups). In another embodiment, the linker is trifunctional (hetero- or homo-). A suitable functional group has reactivity toward or comprises any of the following: amino, alcohol, carboxylic acid, sulfhydryl, alkene, alkyne, azide, thioester, ketone, aldehyde, or hydrazine. An amino acid, e.g., cysteine, can constitute a linker.

According to the invention, x and z are preferably chosen in order to enhance the bioavailability and cutaneous barrier crossing of the compounds, as well as their lipophilicity or lipophilic character. In this case, x and z are preferably a lipophilic moiety.

x and z may also be chosen to promote complementary biological properties of the compound, in addition to the property of the peptidic moiety. The compound of the invention may also, for example, have an anti-inflammatory activity. It may also have anti-oxidant or antiseptic properties.

According to an embodiment of the invention, the compound is selected from the group consisting of x-SR-z, x-FSR-z, x-RFSR-z and x-QRFSR-z, x and z having the above-mentioned definitions.

According to another embodiment of the present invention, z is H or NH₂ and x is a saturated or unsaturated, linear or branched fatty acid containing from 2 to 24 carbon atoms and possibly substituted by one or more heteroatoms.

Preferably, x is a palmitic acid, a stearic acid. Alternatively, x is an omega-3 or an omega-6 fatty acid, more preferably an arachidonic acid.

According to another embodiment of the present invention, z is H and x is an acetic acid.

Preferred compounds of the invention consist in palmitoyl-QRFSR (FIG. 1), palmitoyl-QRFSR-NH₂ (FIG. 2), palmitoyl-NRFSR, palmitoyl-NRFTR, palmitoyl-FSR, acetyl-SR, quercetin-RFSR and arachidonoyl-QRFSR.

The peptide moiety and the compound themselves may be prepared in a conventional manner, by solid phase or liquid phase synthesis. They can also be prepared enzymatic synthesis, by fermentation or by plant or animal extraction.

Solid phase peptide synthesis has been developed by R. B. Merrifield. An insoluble polymer support, also called resin, is used to anchor the peptide chain as each additional alpha-amino acid is attached. A labile group protects the alpha-amino group of the amino acid. This group is removed after each coupling reaction so that the next alpha-amino protected amino acid may be added. Typical labile protecting groups include t-Boc (tert-butyloxycarbonyl) and FMOC (9-fluorenylmethloxycarbonyl). t-Boc is removed with dilute solutions of trifluoroacetic acid (TFA) and dichloromethane. FMOC is removed by concentrated solutions of amines (usually 20-55% piperidine in N-methylpyrrolidone). When using FMOC alpha-amino acids, an acid labile (or base stable) resin, such as an ether resin, is used. The stable blocking group protects the reactive functional group of an amino acid and prevents formation of complicated secondary chains. This blocking group must remain attached throughout the synthesis and may be removed after completion of synthesis. After generation of the resin bound synthetic peptide, the peptide is cleaved from the resin, purified by HPLC if necessary and characterized by HPLC or MALDI.

### Compositions

The composition of the invention is useful for delivering the peptide-based compound to cells or across epithelial and endothelial tissues, such as skin, mucous membranes, vasculature tissues, gastrointestinal tissues, blood brain barrier tissues, ophthalmologic tissues, pulmonary tissues, liver tissues, cardiac tissues, kidney tissues etc. The composition of the invention can be used both for delivery to a particular site of administration or for systemic delivery. Preferably, the composition of the invention is a topical composition.

The composition of the invention can increase delivery or availability of the peptide-based compound to cells or tissues compared to delivery of the peptides described in the prior art, especially the peptides described in EP 1 577 320.

The composition of the invention can either be a cosmetic composition or a pharmaceutical composition.

The composition of the invention is used in a form selected from the group consisting of emulsions (oil-in-water, water-in-oil or oil-in-water-in-oil emulsions), dispersions, solutions, suspensions, liposomes, chylomicrons, nanocapsules, microcapsules, macrocapsules, nanoparticles, microparticles, macroparticles, creams, lotions, ointments, milks, gels, cleansers, foundations, anhydrous preparations (sticks, body and bath oils), shower and bath gels, shampoos and scalp treatment lotions, cream or lotion for care of the skin or hair, sun-screen lotions, milks or creams, artificial suntan lotions, milks, shaving creams or foams, aftershave lotions, make-up, mascaras or nail varnishes, lipsticks, serums, adhesive or absorbent materials, transdermal patches, powders, emollient lotion, emollient milk, emollient cream, sprays, sprayable formulation, oils for the body and the bath, foundation tint bases, pomade, colloid, compact or solid suspension, pencil, brossable, mouthwash, toothpaste tooth liquid gel, an oral or dental care product.

“Liposomes” refer to a membrane composed of a phospholipid and cholesterol bilayer. Liposomes can be composed of naturally-derived phospholipids with mixed lipid chains (e.g. phosphatidylethanolamine), or of pure surfactant components, e.g. DOPE (dioleoylphosphatidylethanolamine).

“Nanocapsules” refer to submicroscopic colloidal drug carrier systems composed of an oily or an aqueous core surrounded by a thin polymer membrane. Two technologies can be used to obtain such nanocapsules: the interfacial polymerization of a monomer or the interfacial nanodeposition of a preformed polymer.

According to the present invention, a “nanoparticle”, also called a nanopowder, a nanocluster or a nanocrystal, refers to a small particle which is less than 100 nm. A “microparticle” refers to a particle which is less than 1 mm. A “macroparticle” refers to a particle which is more than 1 mm.

According to one embodiment of the invention, the composition is a pharmaceutical composition and comprises a pharmaceutically acceptable carrier. The composition of the invention may for example be a dermopharmaceutical composition. As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency, for example listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

According to another embodiment of the invention, the composition is a cosmetic composition, for example a topical, cosmetic composition and comprises a cosmetically acceptable carrier or a dermatologically acceptable carrier. The term “dermatologically acceptable”, as used herein, means that the compositions or components described are suitable for use in contact with human skin without risk of toxicity, incompatibility, instability, allergic response, and the like. The dermatologically acceptable carrier can be an aqueous or hydroalcoholic solution, a water-in-oil emulsion, an-oil-in-water emulsion, a microemulsion, an aqueous gel, an anhydrous gel, a serum or vesicle dispersion. As examples of cosmetic composition, can be mentioned body hygiene compositions, hair compositions, make-up compositions or care compositions. According to this embodiment, the composition of the invention may further comprise at least one additive and/or active agent common in dermocosmetics. Said at least one additive and/or active agent may be chosen from: fragrances; stabilizing agents; UV-A and UV-B screening agents; hydrophilic and lipophilic antioxidants; chelating agents; α- and β-hydroxy acids; ceramides; antidandruff agents; antiacne agents; agents for combating hair loss; antifungal and antiseptic agents; antiperspirant active substances, bactericidal active substances, and odor absorbing active substances; hair fixing material and conditioning material; hair care active agents and sheen-reinforcing agents and hair dyes; fillers, dyes and dyestuffs; gelling material; and silicone gums.

### Therapeutic and Cosmetic Applications

The compounds of the invention demonstrate potency on at least one enkephalin-inactivating ectopeptidase, e.g. human neutral ecto-endopeptidase, hNEP, and/or the human ecto-aminopeptidase, hAP-N. They inhibit pain behavior.

One object of the invention is the use of the above-defined compound for the preparation of a medicament for treating of preventing pain or as an analgesic agent.

Another object of the invention is the use of the above-mentioned compound in a topical cosmetic formulation.

The compound or composition of the invention may notably be used to treat or prevent external aggressions such as insect stings, burns, sunburns, erythema, itches, eczema, psoriasis, wound healing, acute pain, chronic pain polyarthritis and other inflammation pathologies, tumors, infections. The compound of the invention may also promote healing, skin repair, hydro-mineralization of the skin.

Another object of the invention is a process of cosmetically treating human skin or a mucous membrane comprising applying an effective amount of the above-defined topical composition to said skin or mucous membrane.

Another object of the invention is a process of treating a condition comprising applying an effective amount of the above-defined pharmaceutical composition.

### FIGURES

FIG. 1 shows one peptide-based compound according to the invention, the palmitoyl-QRFSR.

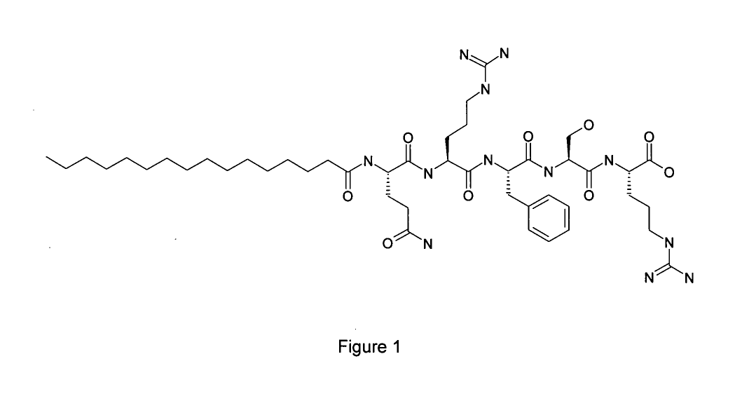

FIG. 2 shows another peptide-based compound according to the invention, the palmitoyl-QRFSR-NH₂.

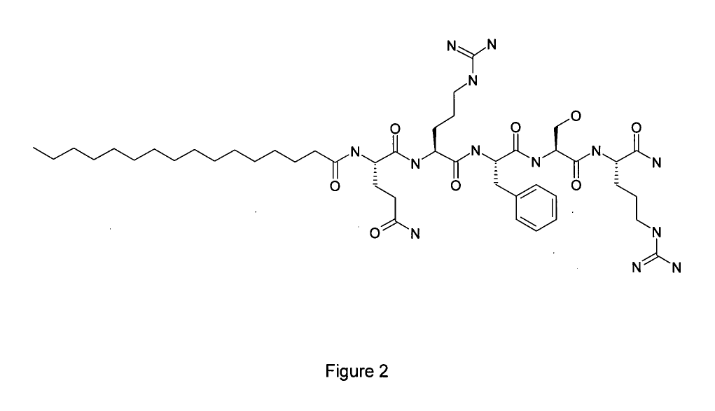

FIG. 3 shows kinetics of Ala-Mca breakdown by recombinant hAP-N in absence of inhibitor (black squares) or in the presence of 6 to 70 μM Pal-QRFSR-NH₂. Each point represents the intensity of the signal expressed in RFU (Relative Fluorescence Unit), which was directly proportional to the quantity of metabolites formed, as function of reaction time (min).

FIG. 4 shows the concentration-dependent inhibition by Pal-QRFSR-NH₂ of Ala-Mca breakdown by pure recombinant human AP-N. Each open square represents the percentage of intact substrate recovered after incubation and calculated as follows: percentage of velocity without inhibitor−velocity in presence of inhibitor/velocity without inhibitor, which was measured in the absence or in the presence of various concentrations of Pal-QRFSR-NH₂ plotted in μM (log-scale).

FIG. 5 shows kinetics of Abz-dRGL-EDDnp breakdown by recombinant hNEP in the presence of corresponding vehicle (black triangles & circles) or in the presence of 3 to 70 μM Pal-QRFSR-NH₂. Each point represents the intensity of the signal expressed in RFU (Relative Fluorescence Unit), which was directly proportional to the quantity of metabolites formed, as function of reaction time (min).

FIG. 6 shows the concentration-dependent inhibition by Pal-QRFSR-NH₂ of Abz-dRGL-EDDnp breakdown by pure recombinant human hNEP. Each open square represents the percentage of intact substrate recovered and calculated as follows: percentage of velocity without inhibitor−velocity in presence of inhibitor/velocity without inhibitor, which was measured in the absence or in the presence of various concentrations of Pal-QRFSR-NH₂ plotted in μM (log-scale).

FIG. 7 shows kinetics of Abz-RGFK-DnpOH breakdown by recombinant hNEP in the absence of inhibitor (black circles) or in the presence of 10 to 60 μM Pal-QRFSR-NH₂. Each point represents the intensity of the signal expressed in RFU (Relative Fluorescence Unit), which was directly proportional to the quantity of metabolites formed, as function of time-reaction (min).

FIG. 8 shows the concentration-dependent inhibition by Pal-QRFSR-NH₂ of Abz-RGFK-DnpOH breakdown by pure recombinant human hNEP. Each point represents the percentage of intact substrate recovered and calculated as follows: percentage of velocity without inhibitor−velocity in presence of inhibitor/velocity without inhibitor, which was measured in the absence or in the presence of various concentrations of tested compound plotted in μM (log-scale).

FIG. 9 shows the kinetic of Mca-RPPGFSAFK-(Dnp)-OH breakdown by recombinant hNEP in the absence of inhibitor (grey triangles & squares) or in the presence of 10 to 70 μM Pal-QRFSR-NH₂. Each point represents the intensity of the signal expressed in RFU (Relative Fluorescence Unit), which was directly proportional to the quantity of metabolites formed, as function of time-reaction (min).

### EXAMPLES

### Example 1

### Synthesis of the Compounds of the Invention

The following compounds have been synthesized: palmitoyl-QRFSR and palmitoyl-QRFSR-NH₂.

The amino acids protected at N-terminal extremity by a group Fmoc (e.g. Fmoc-Gln-OH, Fmoc-Arg(Pbf)-OH, Fmoc-Phe-OH, FmocSer(tBu)-OH) were purchased from SIGMA.

The N,N-dimethylformamide (DMF), the dichloromethane (DCM), methanol, the acetonitrile, ethylic ether, trifluoracetic acid (TFA), the piperidine, were purchased from Carlo Erba or Acros Organics.

The diisopropylcarbodiimide (DIC), the N,N-diisopropylethylamine (DIEA), the N-hydroxybenzotriazole (HOBt), the triisopropylsilane (TIPS), the symmetric anhydre palmitic acid (MW=494) were purchased from SIGMA and Alfa Aesar.

### a) Fmoc-Deprotection

Fmoc deprotection after each amino acid coupling was accomplished using a mixture of DMF/piperidine (80/20, v/v) under agitation during 20 minutes.

### b) Washing

The resin was then washed with DMF (3×5 min), followed by methanol (2×5 min) and successively DCM (2×5 min).

### c) Coupling of Amino Acids

Each amino acid was coupled to the deprotected N-terminal amine of the resin, or previously coupled amino acid, using a coupling solution containing 2 equivalents of amino acid Fmoc, DIC and HOBt in DMF. The coupling solution was added to the deprotected resin during 2 hours.

### d) Monitoring the Progress of Amino Acid Couplings

The progress of amino acid couplings was followed using ninhydrin. The ninhydrin solution turns dark blue (positive result) in the presence of a free primary amine but is otherwise colorless (negative result).

e) Cleaving the Peptide from the Resin

The resin was treated with a solution of TFA/water/TIPS (95/2.5/2.5, v/v/v) for cleavage (5 ml/g, 3 hours). The resin was then filtrated and washed with TFA (5 ml/g). The filtrate was then concentrated under vacuum.

The compounds were precipitated with diethylic ether.

The samples were solubilised, frozen at −80° C. and lyophilized.

### f) Coupling of Palmitate

The palmitate was added by manual coupling. 3 equivalents of palmitic acid (0.33 mmol) were mixed in DIEA (0.4 mmol) and DCM for 3 hours.

### g) Washing

Washing was performed using DCM and NMP. The Kaiser test was negative.

### h) Solubility of the Peptides

The solubilities of the lipopeptides were good in a solution of water/acetonitrile (50/50).

### i) HPLC

The HPLC analyses were performed on Dionex Summit HPLC system, equipped with a P580 pump, a Gina 50 autosampler and a UVD340S diode array detector. The column used is a C18 Merck Chromolith column (100×4.6 mm) thermostated at 35° C. A flow rate of 1 ml/min and a gradient from 20% to 80% of acetonitrile in ammonium formate at 0.1% in 20 minutes were used.

### Example 2

### In Vitro Activity of Palmitoyl-QRFSR-NH₂

The Palmitoyl-QRFSR-NH₂ compound was tested for its inhibitory potency towards two membrane-anchored ectoenzymes that is neutral endopeptidase NEP (EC 3.4.24.11) and aminopeptidase AP-N (EC 3.4.11.2) by using selective fluorescence-based enzyme assays. Wisner et al. (PNAS, 2006) have previously demonstrated that the QRFSR peptide is an efficient dual inhibitor of this two enkephalin-inactivating ectopeptidases.

### A. Biochemical Assays

Formal kinetic analysis was performed for each assay using real-time fluorescence monitoring of specific substrate hydrolysis. For each 96-well adapted fluorimetric model, all parameters allowing the analysis of human NEP and human AP-N enzyme activity were defined under conditions of initial velocity measurement.

1—Sources of the Human Ectopeptidases, hNEP and hAP-N

Recombinant human NEP and recombinant human AP-N (devoid of their respective N-terminal cytosol and transmembrane segment) that were purchased from R&D Systems, were used as pure source of ectopeptidase.

2—Substrates

Aminopeptidase, NEP-carboxydipeptidase and NEP-endopeptidase activities were assayed in vitro by measuring the breakdown of the following synthetic selective substrates:

a) Abz-dR-GL-EDDnp FRET-peptide, an internally quenched fluorescent substrate specific for NEP-endopeptidase activity, was synthesized by Thermo-Fisher Scientific (Germany) (Barros et al., 2007)
b) Abz-RGFK-DnpOH FRET-peptide, an internally quenched fluorescent substrate specific for NEP-carboxydipeptidase activity, was synthesized by Thermo-Fisher Scientific (Germany) (Barros et al., 2007)
c) Mca-RPPGFSAFK-DnpOH FRET-peptide (Mca-BK2), an intramolecularly quenched fluorogenic peptide structurally related to bradykinin and a selective substrate for measuring NEP and ECE activity, was purchased from R&D Systems. ECE, or Endothelin Converting Enzyme, is a mealloectopeptidase enzyme belonging to the same Zn ectoenzyme family than NEP. ECE inactivates the bradykinin and activates the endothelin from its precursor. FRET is the distance-dependant transfer of energy from a donor fluorophore (Abz=ortho-aminobenzoyl; Mca=7-methoxycoumarin-4-yl-acetyl) to an acceptor fluorophore (DnpOH=2,4-dinitrophenyl; EDDnp=2,4-dinitrophenyl ethylenediamine).
d) L-alanine-Mca (or Ala-Mca), a fluorogenic substrate for measuring aminopeptidase activity, was purchased from SIGMA.

3—Measurement of Ectopeptidase Activities Using 96-Well Adapted Fluorimetric Assays

According to conditions of initial velocity measurement: the time, pH and temperature of incubation as well as enzyme and substrate concentrations were defined for each assay. Hydrolysis of peptide-substrates was measured by real-time monitoring their metabolism rate by ectopeptidases in the presence and absence of tested inhibitory compound (concentrations ranging from 1 to 70 μM). These were added to the preincubation medium. The background rate of substrate autolysis representing the fluorescent signal obtained in the absence of enzyme was subtracted to calculate the initial velocities in Relative Fluorescent Unit/min (or RFU/min).

### a) Measurement of NEP-Endopeptidase Activity Using FRET Specific Peptide-Substrate, Abz-dR-GL-EDDnp

Under conditions of initial velocity measurement, the intensity of the signal was directly proportional to the quantity of metabolites formed during the 20-40 min time-period of the reaction. Thus, in absence of inhibitor, the initial velocity of rhNEP-mediated specific endoproteolysis of Abz-dR-GL-EDDnp, was calculated from the linear regression (slope=NEP activity in presence of vehicle/incubation time) as 8218±2878 RFU/min/μg rhNEP, n=3 independent determinations.

### b) Measurement of NEP-Carboxydipeptidase Activity Using FRET Specific Peptide-Substrate Abz-RGFK-DnpOH

Under conditions of initial velocity measurement, human NEP-mediated specific hydrolysis of Abz-RGFK-DnpOH was evaluated at 59796±18685 RFU/min/μg rhNEP, n=4 independent determinations.

In addition, the intramolecularly quenched fluorogenic peptide, Mca-BK2, was submitted to hydrolysis by rhNEP. Under conditions of initial velocity measurement, human NEP-mediated specific hydrolysis of Mca-BK2 was evaluated at 139263±19780 RFU/min/μg rhNEP, n=2 independent determinations.

### c) Measurement of AP-N-Ectopeptidase Activity Using Ala-Mca Substrate.

Under conditions of initial velocity measurement, the human AP-N-mediated aminoproteolysis of Ala-Mca was directly calculated (from the slope: AP-N activity in absence of inhibitor in function of incubation time) as 147042±44657 RFU/min/μg rhAP-N, n=3 independent determinations.

### B. In Vitro Functional Characterization of Palmitoyl-QRFSR-NH₂

The palmitoyl-peptide was dissolved in ethanol at 10 mg/ml (10.7 mM) and stored at −30° C. until it was extemporaneously diluted in the appropriate buffer for testing in each fluorescence-based enzyme-assay. However, as Pal-QRFSR-NH₂ was not soluble in aqueous Tris-buffer conditions at concentration higher than 70 μM, the tested concentrations ranged from 3 to 70 μM.

1—Effect of Palmitoyl-QRFSR-NH, on hAP-N Activity

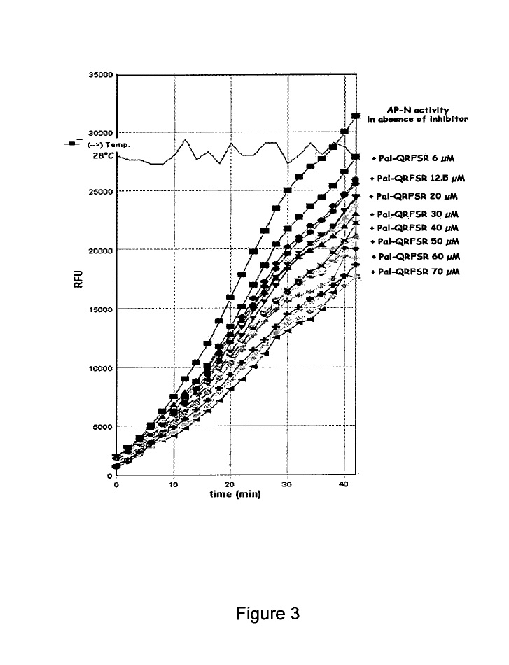

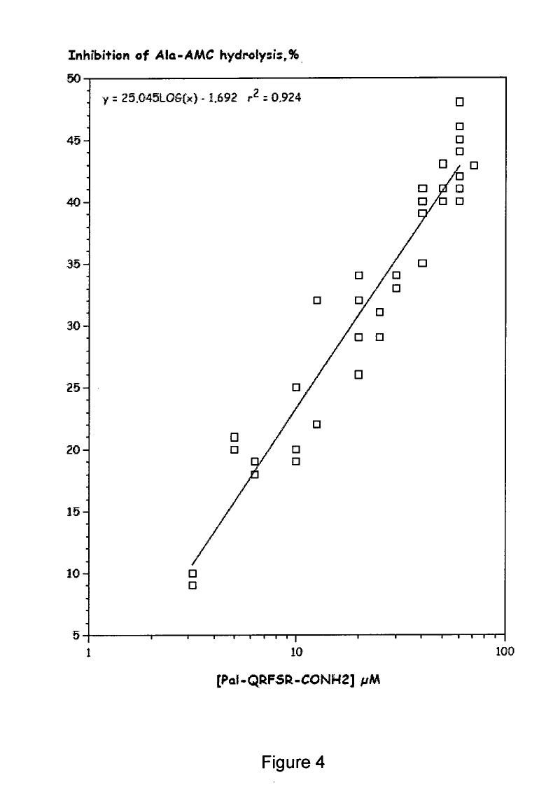

Pal-QRFSR-NH₂ efficiently prevented hAP-N mediated-aminoproteolysis of Ala-Mca substrate. Its inhibitory potency was strictly concentration dependent (r2=0.92, n=34 points of determination); ranging from 3 to 60 μM (FIGS. 3 and 4). Under these experimental conditions, its inhibitory potency was less effective than the QRFSR peptide, but more potent than the pGlu-RFSR peptide. pGlu is pyroglutamate.

Accordingly, the presence of palmitate substitute at the N-terminal moiety of the peptide which is advantageous to prepare a topical formulation, has no negative effects on the recognition by hAP-N and its inhibitory activity.

2—Effect of Palmitoyl-QRFSR-NH₂ on hNEP Endopeptidase Activity

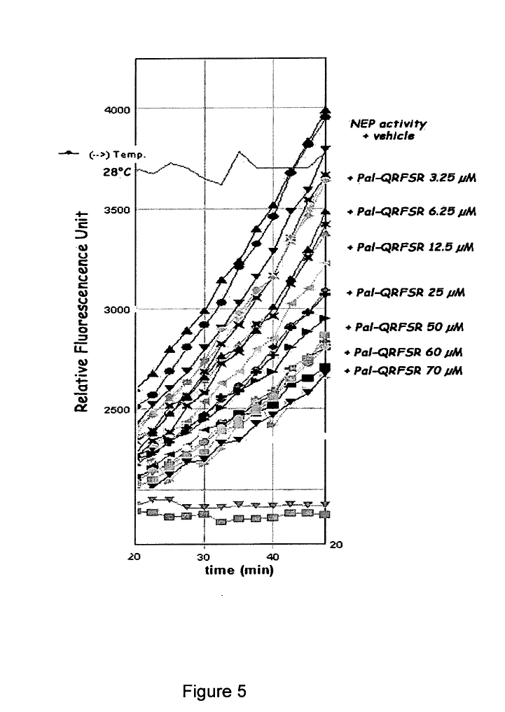

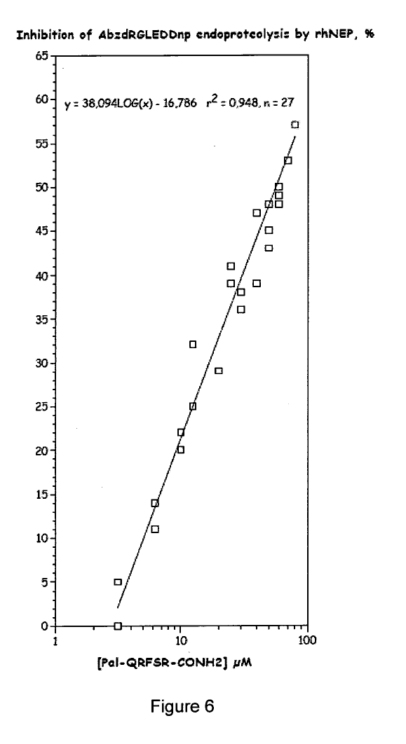

Pal-QRFSR-NH₂ prevented hNEP mediated-endoproteolysis of Abz-dRGL-EDDnp substrate. Its inhibitory potency was strictly concentration dependent (r2=0.95, n=27 points of determination); ranging from 5 to 60 μM (FIGS. 5 and 6). Under these experimental conditions, the compound of the invention demonstrated the same inhibitory potency towards hNEP endopeptidase than the QRFSR.

Accordingly, the presence of palmitate substitute at the N-terminal moiety of the peptide which is advantageous to prepare a topical formulation, has no negative effects on the recognition by hNEP ectoenzyme and its inhibitory activity.

3—Effect of Palmitoyl-QRFSR-NH on hNEP Carboxydipeptidase Activity

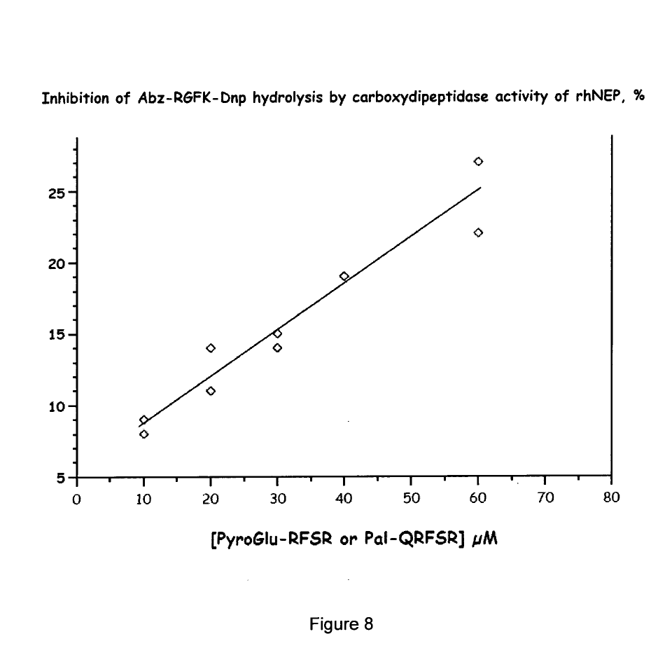

Pal-QRFSR-NH₂ displayed a weak inhibitory potency towards hNEP carboxydipeptidase activity (FIG. 8).

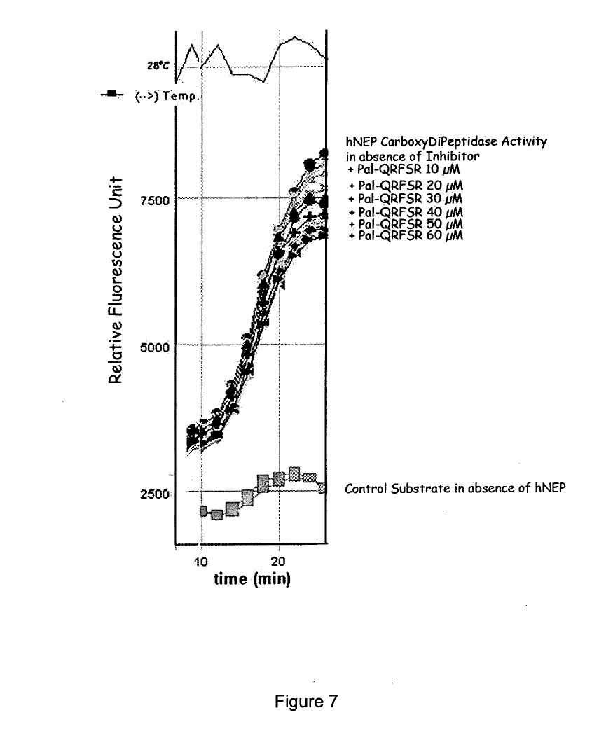

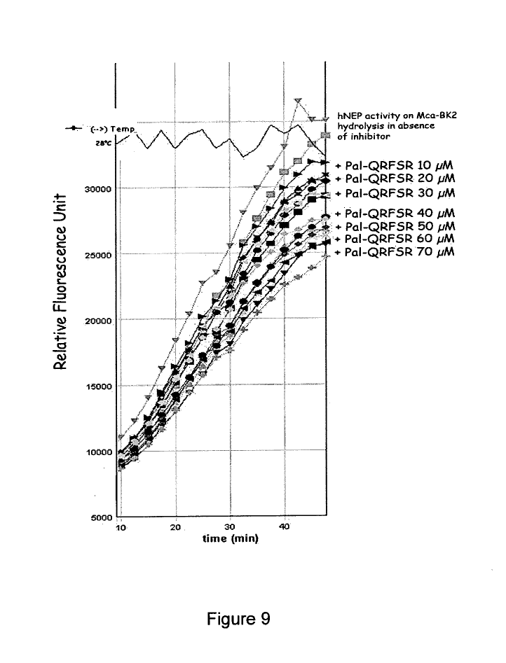

Under the same experimental conditions, Pal-QRFSR-NH₂ displayed a better inhibitory activity on hNEP-mediated hydrolysis of Mca-BK2 substrate (FIG. 9) than on hNEP-mediated hydrolysis of Abz-RGFK-DnpOH substrate (FIG. 7). The latest substrate is particularly specific for carboxydipeptidase activity of NEP, while the Mca-BK2 substrate can be cleaved not only at Phe8 position by carboxydipeptidase activity of NEP but also at Phe5 position by endopeptidase activity of NEP.

### REFERENCES

Barros, N. M., Campos, M., Bersanetti, P. A., Oliveira, V., Juliano, M. A., Boileau, G., Juliano, L. & Carmona, A. K. Neprilysin carboxydipeptidase specificity studies and improvement in its detection with fluorescence energy transfer peptides. Biol Chem (2007) 388, 447-455
Nishimura K, Hazato T (1993) Biochem Biophys Res Commun 194:713-719.
Rougeot C, Messaoudi M, Hermitte V, Rigault A G, Blisnick T, Dugave C, Desor D, Rougeon F. Sialorphin, a natural inhibitor of rat membrane-bound neutral endopeptidase that displays analgesic activity. Proc Natl Acad Sci USA 2003; 100:8549-54.
Wisner, A., et al., Human opiorphin, a natural antinociceptive modulator of opioid-dependent pathways. Proc. Natl. Acad. Sci. USA, (2006).
Yamamoto Y, Ono H, Ueda A, Shimamura M, Nishimura K, Hazato T (2002) Curr Protein Pept Sci 3:587-599.

1. A compound that inhibits a metallo-ectopeptidase, selected in the group consisting of x-aa2-aa1-z, x-aa3-aa2-aa1-z, x-aa4-aa3-aa2-aa1-z and x-aa5-aa4-aa3-aa2-aa1-z, wherein;
aa1 and aa4, independently from each other, represent an arginine amino acid, an histidine amino acid, an asparagine amino acid or a lysine amino acid,
aa2 represents a serine amino acid, a glycine amino acid, a threonine amino acid or an alanine amino acid,
aa3 represents a phenylalanine amino acid, a leucine amino acid, an isoleucine amino acid, a tyrosine amino acid or a tryptophan amino acid,
aa5 represents a glutamine amino acid, a glutamic acid amino acid, an asparagines amino-acid or a lysine amino acid,
x and z, independently from each other, represent:
H;
OH, SH;
a halogen atom,
an amino group,
an alkyl, haloalkyl or heteroalkyl group containing from 1 to 30 carbon atoms, linear or branched,
an alkenyl or alkynyl group containing from 2 to 30 carbon atoms, linear or branched,
an acyl group containing from 1 to 30 carbon atoms, saturated or unsaturated, linear or branched,
a cycloalkyl, cycloakenyl, cycloalkynyl or an heterocyclic group containing from 3 to 30 carbon atoms, linear or branched,
one or more aryl or heteroaryl groups containing from 3 to 10 carbon atoms per cycle,
a alkoxy, thioalkyl, sulfonylalkyl, aminoalkyl containing from 1 to 30 carbon atoms, linear or branched,
one or more heterocyclic group, containing from 5 to 10 carbon atoms per cycle,
said groups being optionally substituted by one or more halogen atoms, alkyl groups, hydroxyl groups, alkoxy groups, aryloxy groups, acyloxy groups, carbamoyloxy groups, carboxy groups, mercapto groups, alkylthio groups, acylthio groups, arylthio groups, aryl groups, heterocyclic groups, heteroaryl groups or amino groups,
with the proviso that when the compound is x-QRFSR-z and z is H, then x is different from H and from a tyrosine amino acid and when the compound is x-RFSR-z and z is H, then x is different a pyroglutamate residue.

2. The compound of claim 1, wherein said peptide is selected from the group consisting of x-SR-z, x-FSR-z, x-RFSR-z and x-QRFSR-z.

3. The compound of claim 1, wherein z is H or NH₂ and x is a saturated or unsaturated, linear or branched fatty acid containing from 2 to 24 carbon atoms and possibly substituted by one or more heteroatoms.

4. The compound of claim 3, wherein x is a palmitic acid or a stearic acid.

5. The compound of claim 3, wherein x is an omega-3 or an omega-6 fatty acid.

6. The compound of claim 5, wherein x is an arachidonic acid.

7. The compound of claim 1, wherein z is H and x is an acetic acid.

8. A composition comprising the compound of claim 1, in association with an acceptable carrier.

9. The composition of claim 8, wherein said composition is a cosmetic or pharmaceutical topical composition.

10. The composition according to claim 9, wherein said topical composition further comprises at least one additive and/or active agent common in dermocosmetics.

11. The composition of claim 8, wherein said composition is used in a form selected from the group consisting of emulsions, dispersions, solutions, suspensions, liposomes, chylomicrons, nanocapsules, microcapsules, macrocapsules, nanoparticles, microparticles, macroparticles, creams, lotions, ointments, milks, gels, cleansers, foundations, anhydrous preparations (sticks, body and bath oils), shower and bath gels, shampoos and scalp treatment lotions, cream or lotion for care of the skin or hair, sun-screen lotions, milks or creams, artificial suntan lotions, milks, shaving creams or foams, aftershave lotions, make-up, mascaras or nail varnishes, lipsticks, serums, adhesive or absorbent materials, transdermal patches, powders, emollient lotion, emollient milk, emollient cream, sprays, sprayable formulation, oils for the body and the bath, foundation tint bases, pomade, colloid, compact or solid suspension, pencil, brossable, mouthwash, toothpaste tooth liquid gel, an oral or dental car product.

12. The composition of claim 8, wherein said compound is present at a concentration between 0.00001% (w/w) and 100% (w/w), preferably at a concentration between 0.0001% (w/w) and 20% (w/w), and more preferably a concentration between 0.001% and 5% (w/w) by weight of the composition.

13. The compound of claim 1, configured for preparation of a medicament for treating of preventing pain.

14. The compound of claim 1, configured in a topical cosmetic formulation.

15. A process of cosmetically treating human skin or a mucous membrane comprising applying an effective amount of the topical composition claim 9 to said skin or mucous membrane.

16. The compound of claim 2, wherein z is H and x is an acetic acid.

17. The composition of claim 10, wherein said composition is used in a form selected from the group consisting of emulsions, dispersions, solutions, suspensions, liposomes, chylomicrons, nanocapsules, microcapsules, macrocapsules, nanoparticles, microparticles, macroparticles, creams, lotions, ointments, milks, gels, cleansers, foundations, anhydrous preparations (sticks, body and bath oils), shower and bath gels, shampoos and scalp treatment lotions, cream or lotion for care of the skin or hair, sun-screen lotions, milks or creams, artificial suntan lotions, milks, shaving creams or foams, aftershave lotions, make-up, mascaras or nail varnishes, lipsticks, serums, adhesive or absorbent materials, transdermal patches, powders, emollient lotion, emollient milk, emollient cream, sprays, sprayable formulation, oils for the body and the bath, foundation tint bases, pomade, colloid, compact or solid suspension, pencil, brossable, mouthwash, toothpaste tooth liquid gel, an oral or dental car product.

18. The composition of claim 9, wherein said compound is present at a concentration between 0.00001% (w/w) and 100% (w/w), preferably at a concentration between 0.0001% (w/w) and 20% (w/w), and more preferably a concentration between 0.001% and 5% (w/w) by weight of the composition.

19. The composition of claim 10, wherein said compound is present at a concentration between 0.00001% (w/w) and 100% (w/w), preferably at a concentration between 0.0001% (w/w) and 20% (w/w), and more preferably a concentration between 0.001% and 5% (w/w) by weight of the composition.

20. The composition of claim 11, wherein said compound is present at a concentration between 0.00001% (w/w) and 100% (w/w), preferably at a concentration between 0.0001% (w/w) and 20% (w/w), and more preferably a concentration between 0.001% and 5% (w/w) by weight of the composition.
